(19) United States
(12) Patent Application Publication
(10) Pub. No.: US 2026/0354844 A1
(43) Pub. Date: Nov. 12, 2026

(54) SURGICAL TOOL FOR TRAJECTORY ADJUSTMENT IN GUIDE PIN INSERTION

(71) Applicant: SUNNYBROOK RESEARCH INSTITUTE, TORONTO (CA)

(72) Inventors: CARI WHYNE, TORONTO (CA); SIERRA MILLER, TORONTO (CA); HANS KREDER, MISSISSAUGA (CA)

(73) Assignee: SUNNYBROOK RESEARCH INSTITUTE, TORONTO (CA)

(21) Appl. No.: 19/661,873

(22) Filed: Apr. 29, 2026

Related U.S. Application Data
(60) Provisional application No. 63/801,888, filed on May 8, 2025.

(51) Int. Cl. A61B 17/17; A61B 6/00

(52) U.S. Cl. CPC A61B 17/1703; A61B 6/485; A61B 17/1717; A61B 17/1746

(57) ABSTRACT

A surgical tool for trajectory adjustment in guide pin insertion includes a conduit extending longitudinally from a proximal end to a distal end. The conduit defines a lumen that extends from a proximal opening at the proximal end to a distal opening at the distal end. A bone anchoring tip is at the distal end of the conduit. The bone anchoring tip has a passage that extends longitudinally therethrough and that is open to the lumen. A trajectory adjustment plate is at the proximal end of the conduit and at least partially covers the proximal opening. The trajectory adjustment plate includes a plurality of laterally spaced apart apertures that extend therethrough and that are open to the lumen. A guide pin is insertable through any one of the apertures into the lumen and from the lumen through the passage. A trajectory of the guide pin on exit from the passage is adjustable by selecting which one of the apertures through which to insert the guide pin.

### CROSS REFERENCES TO RELATED APPLICATIONS

This application claims the benefit of United States Provisional Patent Application No. 63/801,888 filed on May 8, 2025, which is incorporated herein by reference in its entirety.

### FIELD

This document relates to surgical tools, such as those used in pelvic and acetabular reconstructions. More specifically, this document relates to surgical tools for trajectory adjustment in guide pin insertion, and related kits and methods.

### BACKGROUND

U.S. Pat. No. 10,869,679 (Ebrahimi et al.) discloses a positioning and alignment instrument, and methods of use thereof, for facilitating the alignment and insertion of a device, such as a guide wire, into bone. The instrument includes a handheld anchoring component and a rotatable guidance component. During use, the anchoring component is anchored into bone via anchoring protrusions, such that the position and orientation of the anchoring component is fixed relative to the bone. The guidance component, which is mechanically supported by the anchoring component, includes a device guide channel for receiving the device and guiding the device towards an insertion location adjacent to the distal end of the anchoring component. The guidance component is rotatable relative to the anchoring component about a rotation axis that is located adjacent to the distal end of the anchoring component, such that the insertion location remains adjacent to the distal end of the anchoring component under rotation.

### SUMMARY

The following summary is intended to introduce the reader to various aspects of the detailed description, but not to define or delimit any invention.

Surgical tools are disclosed. According to some aspects, a surgical tool for trajectory adjustment in guide pin insertion includes a conduit extending longitudinally from a proximal end to a distal end. The conduit defines a lumen that extends from a proximal opening at the proximal end to a distal opening at the distal end. A bone anchoring tip is at the distal end of the conduit. The bone anchoring tip has a passage that extends longitudinally therethrough and that is open to the lumen. A trajectory adjustment plate is at the proximal end of the conduit and at least partially covers the proximal opening. The trajectory adjustment plate includes a plurality of laterally spaced apart apertures that extend therethrough and that are open to the lumen. A guide pin is insertable through any one of the apertures into the lumen and from the lumen through the passage, and whereby a trajectory of the guide pin on exit from the passage is adjustable by selecting which one of the apertures through which to insert the guide pin.

In some examples, the apertures are arranged in a grid. In some examples, the trajectory adjustment plate includes at least 9 of the apertures. In some examples, the apertures all extend parallel to each other.

In some examples, the bone anchoring tip includes a plurality of distally extending teeth for engaging with a bone surface.

In some examples, the conduit includes at least a first radiopaque marker to facilitate positioning of the surgical tool using fluoroscopy. The conduit may include at least a portion that is generally polygonal in transverse section to define at least a first surface that is generally planar, and the first surface may provide the first radiopaque marker. A proximal portion of the conduit may be generally polygonal in transverse section, and a distal portion of the conduit may be generally circular in transverse section.

In some examples, the conduit tapers in cross-sectional area going from the proximal end towards the distal end.

In some examples, the surgical tool further includes a handle mounted to the conduit for manipulation of the surgical tool.

In some examples, the conduit is fabricated from a plastic. In some examples, the trajectory adjustment plate is fabricated from a metal. In some examples, the bone anchoring tip is fabricated from a metal.

Kits of parts for guide pin insertion are also disclosed. According to some aspects, a kit of parts for guide pin insertion includes a surgical tool and a guide pin. The surgical tool includes a conduit extending longitudinally from a proximal end to a distal end, wherein the conduit defines a lumen that extends from a proximal opening at the proximal end to a distal opening at the distal end; a bone anchoring tip at the distal end of the conduit, wherein the bone anchoring tip has a passage that extends longitudinally therethrough and that is open to the lumen; and a trajectory adjustment plate at the proximal end of the conduit and at least partially covering the proximal opening, wherein the trajectory adjustment plate includes a plurality of spaced apart apertures that extend therethrough and that are open to the lumen. The guide pin is insertable through any one of the apertures into the lumen and from the lumen through the passage. A trajectory of the guide pin on exit from the passage is adjustable by selecting which one of the apertures through which to insert the guide pin.

In some examples, the apertures are arranged in a grid. In some examples, the trajectory adjustment plate includes at least 9 of the apertures. In some examples, the apertures all extend parallel to each other.

In some examples, the bone anchoring tip includes a plurality of distally extending teeth for engaging with a bone surface.

In some examples, the conduit includes at least a first radiopaque marker to facilitate positioning of the surgical tool using fluoroscopy. The conduit may include at least a portion that is generally polygonal in transverse section to define at least a first surface that is generally planar, and the first surface may provide the first radiopaque marker. A proximal portion of the conduit may be generally polygonal in transverse section, and a distal portion of the conduit may be generally circular in transverse section.

In some examples, the conduit tapers in cross-sectional area going from the proximal end towards the distal end.

In some examples, the surgical tool further includes a handle mounted to the conduit for manipulation of the surgical tool.

In some examples, the conduit is fabricated from a plastic. In some examples, the trajectory adjustment plate is fabricated from a metal. In some examples, the bone anchoring tip is fabricated from a metal.

Methods for adjusting a trajectory of a guide pin are also disclosed. According to some aspects, a method for adjusting a trajectory of a guide pin includes: a. positioning a bone anchoring tip of a surgical tool against a bone; b. concurrently with or after step a., inserting a guide pin longitudinally through the surgical tool such that the guide pin extends through a first aperture of a trajectory adjustment plate of the surgical tool and into a lumen of a conduit of the surgical tool, through the lumen and into the bone anchoring tip, and through the bone anchoring tip and into the bone; c. using fluoroscopy to assess a trajectory of the guide pin into the bone; and d. adjusting the trajectory by adjusting the guide pin to go through a different (second) aperture of the trajectory adjustment plate, whereby the second aperture is spaced laterally from the first aperture.

In some examples, step d. includes removing the guide pin from the bone and the surgical tool, and re-inserting the guide pin through the surgical tool by passing the guide pin through the second aperture of the trajectory adjustment plate and into the lumen, through the lumen and into the passage of the bone anchoring tip, and through the passage and into the bone.

In some examples, step d. includes removing the trajectory adjustment plate from the surgical tool and from the guide pin, repositioning the trajectory adjustment plate such that the guide pin is inserted through the second aperture of the trajectory adjustment plate, and reattaching the trajectory adjustment plate to the surgical tool.

In some examples, step a. includes engaging distal teeth of the bone anchoring tip with a surface of the bone.

In some examples, the method further includes: e. using fluoroscopy to re-assess the trajectory of the guide pin into the bone; and f. re-adjusting the trajectory of the guide as per step d. through a third aperture of the trajectory adjustment plate, whereby the third aperture is spaced laterally from the first aperture and second aperture. This readjustment can be repeated until the desired trajectory is achieved.

In some examples, step c. includes assessing the trajectory of the guide pin in a first plane, and step e. comprises re-assessing the trajectory of the guide pin in a second plane that is perpendicular to the first plane.

In some examples, the second aperture is spaced from the first aperture in a first lateral direction, and the third aperture is spaced from the second aperture in a second lateral direction that is transverse to the first direction.

In some examples, the method further includes: g. viewing at least one surface of the conduit under fluoroscopy.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a perspective view of an example surgical tool, showing a guide pin inserted through the surgical tool in a first position (in solid line) and in a second position (in dotted line);

FIG. 2 is a side view of the surgical tool of FIG. 1;

FIG. 3 is a rear view of the surgical tool of FIG. 1;

FIG. 4 is a front view of the surgical tool of FIG. 1;

FIG. 5 is a cross section taken along line 5 -5 in FIG. 3;

FIG. 6 is a schematic drawing showing a step of a method for using the surgical tool and guide pin of FIG. 1;

FIG. 7 is a schematic drawing showing another step of the method of FIG. 6;

FIG. 8 is a schematic drawing showing yet another step of the method of FIG. 6; and

FIG. 9 is a schematic drawing showing yet another step of the method of FIG. 6.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof. Furthermore, the phase “at least one of A and B” is intended to mean only A (i.e. one or multiple of A), only B (i.e. one or multiple of B), or a combination of one or more of A and one or more of B.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Generally disclosed herein is a surgical tool for trajectory adjustment in guide pin insertion, and related kits of parts and methods. The surgical tool can be used, for example, in pelvic and acetabular reconstruction, neurosurgery, and/or spinal surgery. More specifically, the surgical tool can be used, for example, in procedures involving the insertion of anterior or posterior column screws, supra and infra acetabular corridor screws for acetabular fractures, the insertion of iliosacral or transsacral screws for sacroiliac joint disruption or sacral fractures, or the insertion of pubic ramus screws for ramus fractures.

The above-noted procedures may involve the insertion and positioning of a guide pin (also known as a guide wire) in a pilot hole in a bone, with the guide pin oriented such that the trajectory of the guide pin passes through a narrow bone corridor while avoiding non-target structures (e.g. neurological and vascular structures). As used herein, the term “trajectory” refers to the path that the guide pin would follow if insertion was continued in a generally straight line. Once that trajectory is determined to be satisfactory with appropriate imaging (as described below), the guide pin is advanced across the injured structures to the intended termination within the bone. The length of the guidewire within the bone is then measured and the appropriate length cannulated screw is selected based on this measurement. Finally, the selected length screw is inserted over the guide pin such that the screw, once fully inserted, passes through the narrow bone corridor and avoids the non-target structures.

The insertion and positioning of the guide pin may be carried out with the aid of 2-dimensional (2D) fluoroscopic imaging. More specifically, using fluoroscopic imaging, the guide pin may be positioned in a pilot hole such that it provides the desired trajectory in one plane. The X-ray unit may then be moved to the perpendicular plane, and the position of the guide pin in the perpendicular plane may then be adjusted to provide the desired trajectory in the perpendicular plane. Once the desired trajectory is achieved in both planes, the guide pin may be advanced across the injured structures to the desired termination position. A cannulated screw may then be inserted over the guide pin across the injured structures.

The surgical tool disclosed herein can be used to facilitate positioning and subsequent insertion of the guide pin to provide the desired trajectory. In particular, the surgical tool disclosed herein may allow for discrete adjustments in the positioning of the guide pin, to thereby provide discrete adjustments in the trajectory of the guide pin. For example the surgical tool may allow for a user to select an adjustment in a desired plane, and to select adjustment on the scale of millimeters of movement. Furthermore, the surgical tool can allow for adjustment of the trajectory of the guide pin in one desired plane at a time, such that adjustments in any one plane do not result in the loss of the desired trajectory in the other plane. This can simplify surgical procedures, and result in decreased surgical time and radiation exposure.

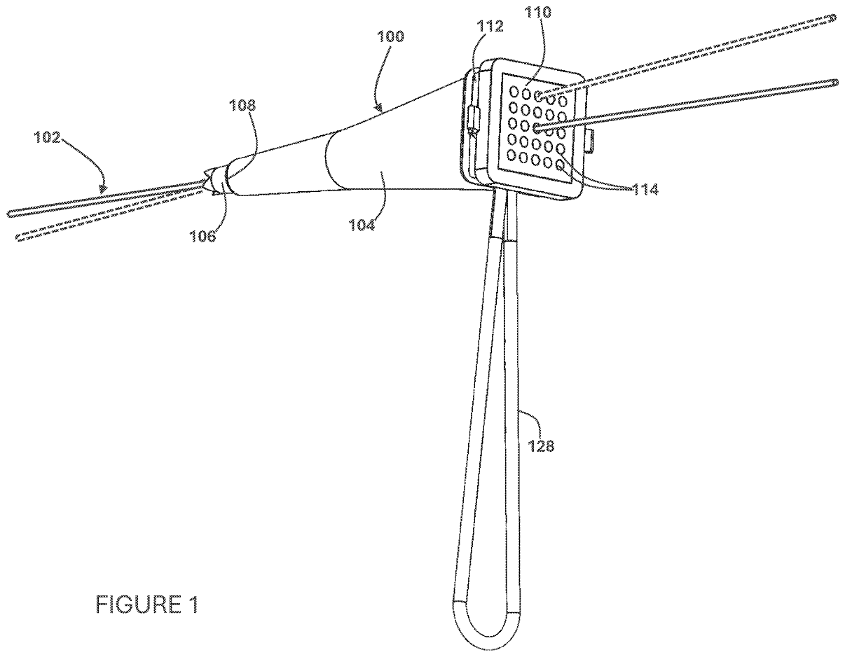

Referring now to FIG. 1, an example surgical tool 100 is shown. The surgical tool 100 is shown together with a guide pin 102. The guide pin 102 may be any suitable guide pin, such as a stainless steel guide pin having a diameter of between about 2.5 mm and 2.8 mm. Guide pins are well known in the art and will not be described in detail herein. Furthermore, the surgical tool 100 may be provided together with the guide pin 102 (e.g. sold as a kit), or may be provided separately.

Referring still to FIG. 1, the surgical tool 100 generally includes a conduit 104 having a bone anchoring tip 106 at a distal end 108 thereof, and a trajectory adjustment plate 110 at a proximal end 112 thereof. The trajectory adjustment plate 110 includes a plurality of apertures 114 (only two of which are labelled in FIG. 1). In use, the guide pin 102 may be inserted through any one of the apertures 114 of the trajectory adjustment plate 110 and into the conduit 104, and from the conduit 104 through the bone anchoring tip 106, to engage with a patient's bone (not shown in FIG. 1). In FIG. 1, the guide pin 102 is shown in solid line in a first position with respect to the surgical tool 100, having been inserted through a first of the apertures 114 of the trajectory adjustment plate 110. Furthermore, the guide pin 102 is shown in dotted line in a second position with respect to the surgical tool 100, having been inserted through a second of the apertures 114 of the trajectory adjustment plate 110. As will be described in further detail below, by adjusting the guide pin 102 from the first position to the second position, the trajectory of the guide pin is adjusted and this adjustment can be limited to a single plane that is perpendicular to the two dimensional fluoroscopic image used to guide insertion trajectory without affecting the other plane. In other words, the trajectory of the guide pin 102 is adjustable by selecting which one of the apertures 114 through which to insert the guide pin 102.

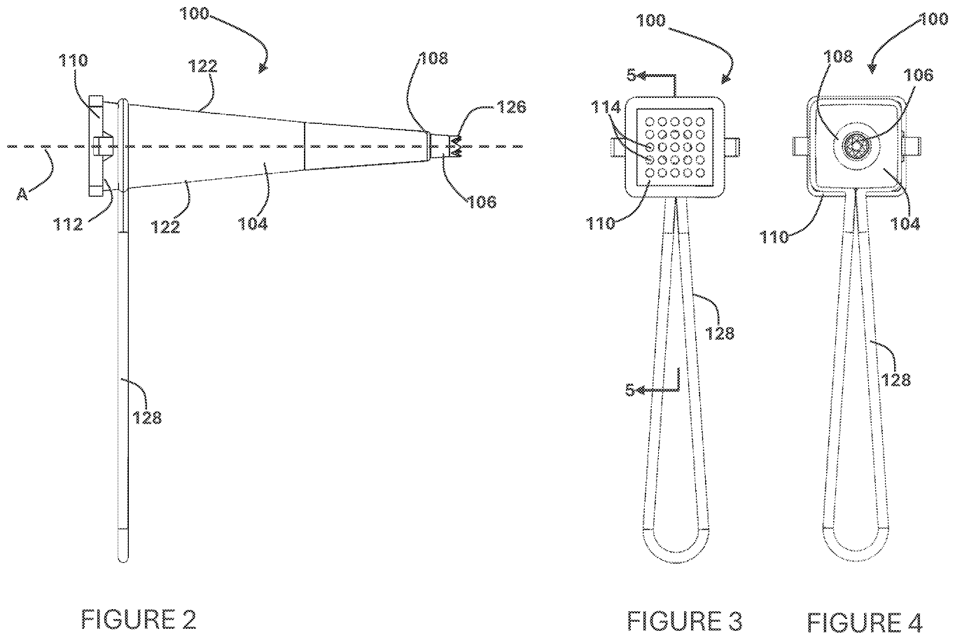

Referring now to FIGS. 2 to 5, the parts of the surgical tool 100 will be described in further detail.

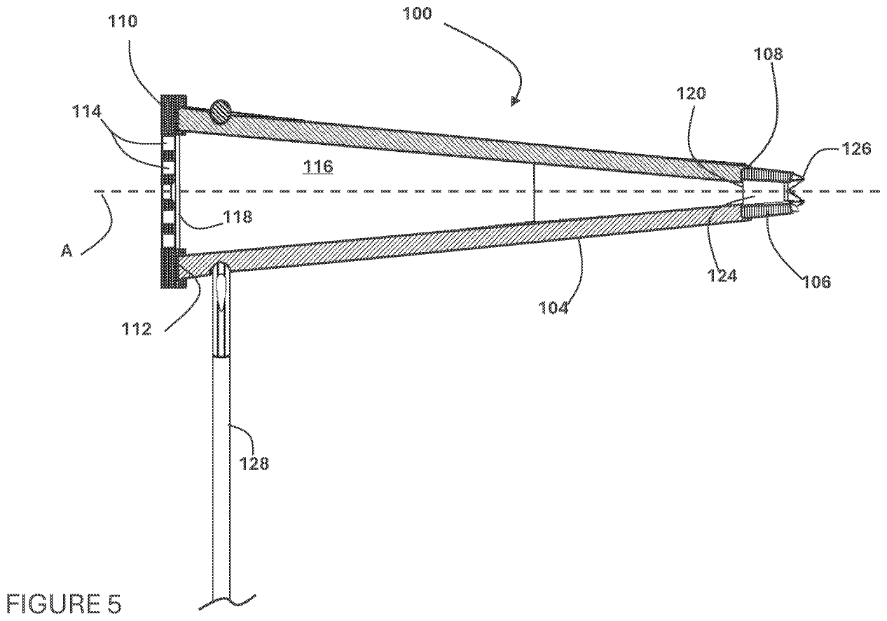

The conduit 104 generally serves to hold (i.e. mechanically support) the bone anchoring tip 106 and the trajectory adjustment plate 110 in place at opposite ends of the tool 100, while providing a corridor for passage of the guide pin 102 (not shown in FIGS. 2 to 5) from the trajectory adjustment plane 110 to the bone anchoring tip 106. In the example shown, the conduit 104 extends longitudinally along longitudinal axis A (shown in FIGS. 2 and 5) from the proximal end 112 thereof to the distal end 108 thereof. As shown in FIG. 5, the conduit 104 defines a lumen 116 that extends from a proximal opening 118 at the proximal end 112 to a distal opening 120 at the distal end 108.

In the example shown, the conduit 104 tapers in cross-sectional area going from the proximal end 112 towards the distal end 108, such that the distal opening 120 is smaller than the proximal opening 118. Accordingly, while the guide pin 102 may enter the proximal opening 118 at various locations via apertures 114 in the trajectory adjustment plate 110, it may exit the distal opening 120 at generally only a single point. As such, adjustment of the position of the guide pin 102 with respect to the surgical tool 100 results in a change incline of the guide pin 102 with respect to the longitudinal axis A.

In the example shown, the conduit 104 is a generally solid-walled tubular structure. In other examples, the conduit 104 may be of another configuration that serves to hold the bone anchoring tip 106 and the trajectory adjustment plate 110 in place at opposite ends of the surgical tool 100, and through which the guide pin 102 may be inserted.

The conduit 104 may include one or more radiopaque markers to facilitate positioning of the surgical tool 100 under fluoroscopy. In the example shown, a portion of the conduit 104 is generally polygonal (i.e. square in the example shown) in transverse section. More specifically, a proximal portion of the conduit 104 is polygonal in transverse section, while a distal portion of the conduit 104 is generally circular in transverse section. The polygonal proximal portion defines a set of surfaces 122 (i.e. first through fourth surfaces, two of which are labelled in FIG. 2) that are generally planar. These surfaces 122 may visible under fluoroscopy when viewed in the orthogonal plane, and may serve as a set of radiopaque markers (i.e. first through fourth radiopaque markers). This allows the guide pin trajectory to be adjusted perpendicular to the fluoroscopic image without affecting the trajectory in the other plane.

The conduit 104 may be fabricated from a variety of materials, such as plastic (e.g. polyetheretherketone (PEEK), lucite, and/or polycarbonates). In examples in which the surfaces 122 of the conduit 104 serve as radiopaque markers, the conduit may be made from a relatively radiolucent material, such that generally only the surfaces 122 are visible under fluoroscopy, and generally only when viewed in the orthogonal plane.

The conduit 104 may, for example, have a length of between 100 mm and 200 mm (e.g. about 154.4 mm). The distal opening 120 may have a diameter of, for example, between 2.5 mm and about 7.5 mm (e.g. about 5.4 mm). The proximal opening 118 may be generally square and may for example have a side length of between about 25 mm and about 50 mm (e.g. about 35 mm).

Referring still to FIGS. 2 to 5, the bone anchoring tip 106 is at the distal end 108 of the conduit 104, and serves to engage with a patient's bone to anchor the surgical tool 100 to the bone. For example, the bone anchoring tip 106 may rest against a surface of a bone, or may inserted into a pilot hole in the bone.

In the example shown, the bone anchoring tip 106 is generally cylindrical and has a passage 124 that extends longitudinally therethrough along axis A. The passage 124 is open to the lumen 116, such that the guide pin 102 may pass from the lumen 116 and through the passage 124.

In the example shown, the bone anchoring tip 106 includes a plurality of distally extending teeth 126 (only one of which is labelled), for engaging with a bone surface. In use, the bone anchoring teeth 126 may dig or bite into the surface of the bone, to prevent slipping of the surgical tool 100 along the bone.

The bone anchoring tip 106 may, for example, be fabricated from a metal such as stainless steel.

The bone anchoring tip 106 may, for example, be mounted to the conduit 104 by friction fit, by snap fit, by screw threads, by an adhesive, or in another suitable fashion. Alternatively, the bone anchoring tip 106 may be integral with the conduit 104.

The bone anchoring tip 106 may taper in diameter going in the distal direction. For example, the passage 124 may for example taper from a diameter of between about 5 mm and about 6 mm (e.g. about 5.4 mm) to a diameter of between about 3.5 mm and about 4.5 mm (e.g. about 4 mm). The bone anchoring tip 106 may, for example, have a length of between about 15 mm and about 25 mm (e.g. about 19.4 mm).

Referring still to FIGS. 2 to 5, the trajectory adjustment plate 110 is at the proximal end 112 of the conduit 104, and serves to facilitate adjustment in the positioning of the guide pin 102, in order to adjust the trajectory of the guide pin 102.

In the example shown, the trajectory adjustment plate 110 is mounted to the conduit 104 to generally cover the proximal opening 118 (i.e. to fully or partially cover the proximal opening 118). The apertures 114 extend through the trajectory adjustment plate 110 and are open to the lumen 116, so that a guide pin 102 may be inserted through any one of the apertures 114 and into the lumen 116. The apertures 114 are laterally spaced apart (i.e. spaced apart in directions that are perpendicular to the longitudinal axis A), so that by selecting which of the apertures 114 through which to insert the guide pin 102, a trajectory of the guide pin 102 on exit from the passage 124 of the bone anchoring tip 106 is adjusted (as shown in FIG. 1, and as will be described in further detail below).

In the example shown, the trajectory adjustment plate 110 includes 25 of the apertures 114, which are arranged in a 5 by 5 grid. In alternative examples, the trajectory adjustment plate 110 may include another number of the apertures 114, such as 9 of the apertures 114, 49 of the apertures 114, or more than 49 of the apertures 114, or any other number of the apertures 114. In further alternative examples, the apertures 114 may be arranged in another fashion, such as in concentric circles.

Preferably, the apertures 114 are laterally spaced apart in two directions. For example, when viewed from the rear as in FIG. 3, the apertures 114 are laterally spaced apart both horizontally and vertically. This can facilitate adjustment in two directions. However, in alternative examples, the apertures 114 may be spaced apart in only one direction (e.g. they may be arranged in a single line).

In the example shown, the apertures 114 all extend parallel to each other and parallel to the longitudinal axis A. In alternative examples, one or more of the apertures 114 may be non-parallel to the other apertures 114 and/or non-parallel to the longitudinal axis A, to further adjust the trajectory of the guide pin 102.

The apertures 114 may be sized so that they are slightly larger than the guide pin 102, to allow for the guide pin 102 to pass therethrough when it is parallel to the longitudinal axis A and when it is inclined with respect to the longitudinal axis A. For example, the apertures may have a diameter of between about 2.5 mm and about 3 mm. More specifically, in instances where the guide pin has a diameter of 2.5 mm. the apertures may have a diameter of between about 2.6 mm and about 3 mm.

In the example shown, the apertures 114 are discrete (i.e. are not connected to each other), and movement of a guide pin 102 from one aperture 114 to the next is achieved by removal of the guide pin 102 from the surgical tool 100 and reinsertion of the guide pin 102 through the surgical tool 100. In alternative examples, the apertures 114 may be connected by slots, so that the guide pin 102 may be snapped from one aperture 114 to the next.

The trajectory adjustment plate 110 may, for example, be mounted to the conduit 104 by slotted fit, friction fit, magnetic forces, snap fit, or in another fashion. In some examples, the trajectory adjustment plate 110 is removable from the conduit 104. Alternatively, the trajectory adjustment plate 110 may be integral with the conduit 104.

The trajectory adjustment plate 110 may, for example, be fabricated from a metal such as stainless steel, or a plastic such as PEEK.

In the example shown, the trajectory adjustment plate 110 is generally square, and may have a length and width of, for example, between about 10 mm and about 50 mm (e.g. about 15 mm, or about 30 mm, or about 45 mm). In alternative examples, the trajectory adjustment plate 110 may be another shape (e.g. rectangular or circular).

Referring still to FIGS. 2 to 5, in the example shown, the surgical tool 110 further includes a handle 128 for manipulation of the surgical tool 100. The handle 128 is mounted to the conduit.

In the example shown, the conduit 104, bone anchoring tip 106, trajectory adjustment plate 110, and handle 128 are made from separate parts that are mounted together. In alternative examples, the surgical tool 100 may be all one piece, or certain of the parts may be integral with each other.

The surgical tool 100 may be considered a single use device that is disposable, or may be sterilizable and used multiple times.

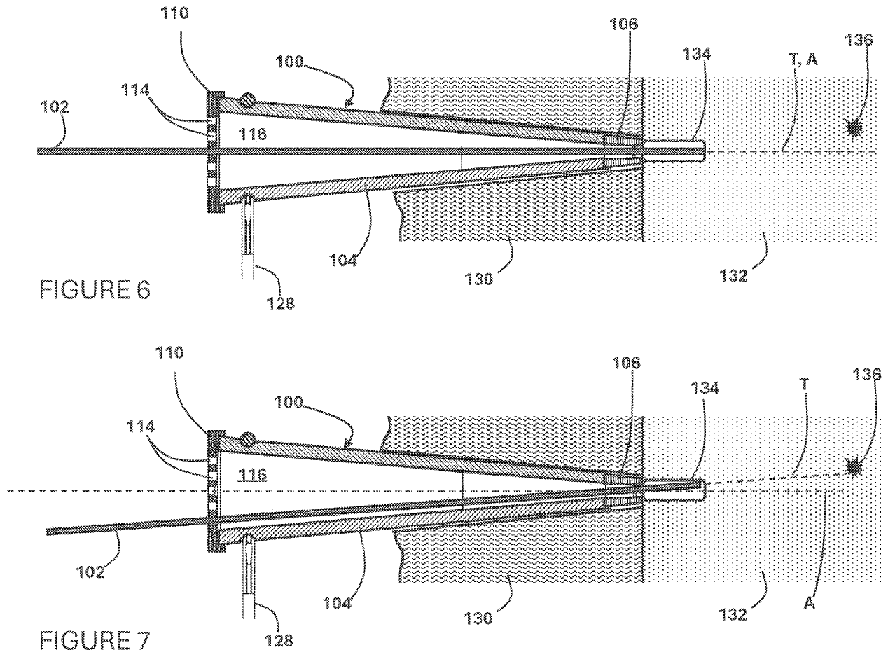

Referring now to FIGS. 6 to 9, an example method for adjusting the trajectory of a guide pin will now be described. The method will be described with reference to the surgical tool 100 and guide pin 102 described above; however, the surgical tool 100 is not limited to use according to the described method, and the method is not limited to the described surgical tool 100 and guide pin 102. Furthermore, FIG. 6 to 9 are schematic drawings, which generally show a patient's anatomy and positioning of the surgical tool 100 and guide pin 102 in use. FIGS. 6 to 9 are intended to generally and schematically represent fluoroscopic views of the surgical tool 100, guide pin 102, and patient's anatomy; however, it will be appreciated that in use under fluoroscopy, only certain parts of the guide pin 102, surgical tool 100, and anatomy would be visible.

Referring first to FIG. 6, a surgical site is shown schematically, including a patient's soft tissue 130, a bone 132 with a pilot hole 134 drilled or otherwise formed therein, and target location 136 within the bone. For example, the soft tissue 130 may be the patient's buttocks, the bone 130 may be the ilium, and the target location may be the sacrum. Once the surgical site has been prepared, the surgical tool 100 may be inserted through the soft tissue 130 and towards the pilot hole 134, such that the bone anchoring tip 106 is against the surface of the bone 132, surrounding the pilot hole 134, with the distal teeth 126 (not labelled in FIGS. 6 to 9) engaging with the surface of the bone 132. This can optionally be facilitated with the use of a relatively small wire (commonly referred to as a “K wire”), which can initially be inserted through a starting point in the skin, and over which the surgical tool 100 can be inserted, following which the wire can be removed. The surgical tool 100 may be held in place using the handle 128; however, the surrounding soft tissue 130 may also serve to hold the surgical tool 100 in place. Fluoroscopy may be used to assess the position of the surgical tool 100 with respect to the bone 132, by viewing one or more surfaces 122 (not labelled in FIGS. 6 to 9) of the surgical tool 100 under fluoroscopy.

The guide pin 102 may then be inserted through longitudinally through the surgical tool 100 by passing the guide pin 102 through a first of the apertures 114 of the trajectory adjustment plate 110 and into the lumen 116 of the conduit 104; through the lumen 116 and into the passage 124 (not visible in FIGS. 6 to 9) of the bone anchoring tip 106; and through the passage 124 and into the pilot hole 134 in the bone 132. The first of the apertures may be any one of the apertures 114, and is selectable by the user (e.g. the surgeon); however, in the example shown, the first of the apertures 114 is centrally located, such that the guide pin 102 is generally inserted along the longitudinal axis A of conduit 104.

In an alternative example (not shown), the guide pin may be inserted into the pilot hole prior to the insertion of the surgical tool. Then, the surgical tool may be positioned in the patient's tissue concurrently with the insertion of the guide pin through the surgical tool. For example, the trajectory adjustment plate may be removed from the surgical tool, and the surgical tool may be slid over the guide pin until the bone anchoring tip is against the surface of the bone, and such that such that the guide pin extends through the conduit and through the bone anchoring tip. The trajectory adjustment plate may then be reattached to the surgical tool, with the guide pin optionally in the centre aperture.

The position of the guide pin 102 may then be assessed using fluoroscopy in a first plane, to assess the trajectory of the guide pin 102 into the bone in the first plane. The trajectory is generally shown at line T in FIGS. 6 to 9 (and in FIG. 6, the trajectory T and longitudinal axis A are one and the same). It will be appreciated that the trajectory T is an imaginary line that is not visible in use; the trajectory T is assessed by viewing the guide pin under fluoroscopy and considering, for example, the angle of the guide pin and the direction in which the guide pin is pointing. When the guide pin 102 is in the position shown in FIG. 6 and is viewed under fluoroscopy in the first plane, the user would determine that the guide pin 102 does not provide the desired trajectory T in the first plane, as the trajectory T does not intersect the target location 136. In other words, under fluoroscopy, it would be apparent to the user that the guide pin does not point towards the target location 134; thus, if a bone screw were inserted over the guide pin 102 to follow the trajectory T of the guide pin as shown in FIG. 6, the bone screw would not be in the correct position. Accordingly, the position of the guide pin 102 should be adjusted in order to adjust the trajectory T in the first plane.

Referring now to FIG. 7, in order to adjust the trajectory T in the first plane, the user may remove the guide pin 102 from the pilot hole 134 in the bone 132 and from the surgical tool 100, and re-insert the guide pin 102 through the surgical tool 100 and into the pilot hole 134 in the bone 132. In particular, the user may pass the guide pin 102 through a second of the apertures 114 of the trajectory adjustment plate 110 and into the lumen 116 of the conduit 104; through the lumen 116 and into the passage 124 of the bone anchoring tip 106; and through the passage 124 and into the pilot hole 134 in the bone 132. Alternatively (not shown), the user may leave the guide pin 102 and surgical tool 100 in place, and remove the trajectory adjustment plate 110 from the surgical tool 100 and guide pin 102. The user may then reposition the trajectory adjustment plate 110 such that the guide pin 102 is inserted through a second one of the apertures, and reattach the trajectory adjustment plate 110 to the surgical tool 100.

The second of the apertures 114 may be selected by the user; however, in the example shown, the second of the apertures 114 is located in a bottom row of the apertures 114, and is vertically aligned with the first aperture 114, such that the repositioning from the first of the apertures 114 to the second of the apertures 114 adjusts the trajectory T in only the first plane. In the example shown, the movement of the guide pin 102 from the first of the apertures 114 to the second of the apertures 114 has resulted in a relatively large adjustment; however, if desired, the user may opt for a smaller adjustment by selecting a second aperture 114 that is closer to the first aperture 114.

As shown in FIG. 7, the repositioning of the guide pin 102 from the first of the apertures 114 to the second of the apertures 114 results in the trajectory T of the guide pin 102 pointing generally upwards as compared to the trajectory T shown in FIG. 6. In other words, the repositioning of the guide pin 102 using the surgical tool 100 has resulted in a change in the incline of the guide pin 102 with respect to the longitudinal axis A of the conduit 104.

The position of the guide pin 102 may then be re-assessed using fluoroscopy, to re-assess the trajectory T of the guide pin 102 into the bone 132 in the first plane. When the guide pin 102 is in the position shown in FIG. 7 and is viewed under fluoroscopy, the user would determine that the guide pin 102 does provide the desired trajectory T in the first plane, as the trajectory T intersects the target location 136. In other words, under fluoroscopy, it would be apparent to the user that the guide pin 102 points towards the target location 136; thus, if a bone screw were inserted over the guide pin 102 to follow the trajectory T of the guide pin as shown in FIG. 7, the bone screw would be in the correct position in the first plane.

The steps of re-positioning the guide pin 102 and re-assessing the position of the guide pin 102 may be repeated as many times as desired or as necessary (i.e. by selecting subsequent apertures 114), until the guide pin 102 provides the desired trajectory T in the first plane.

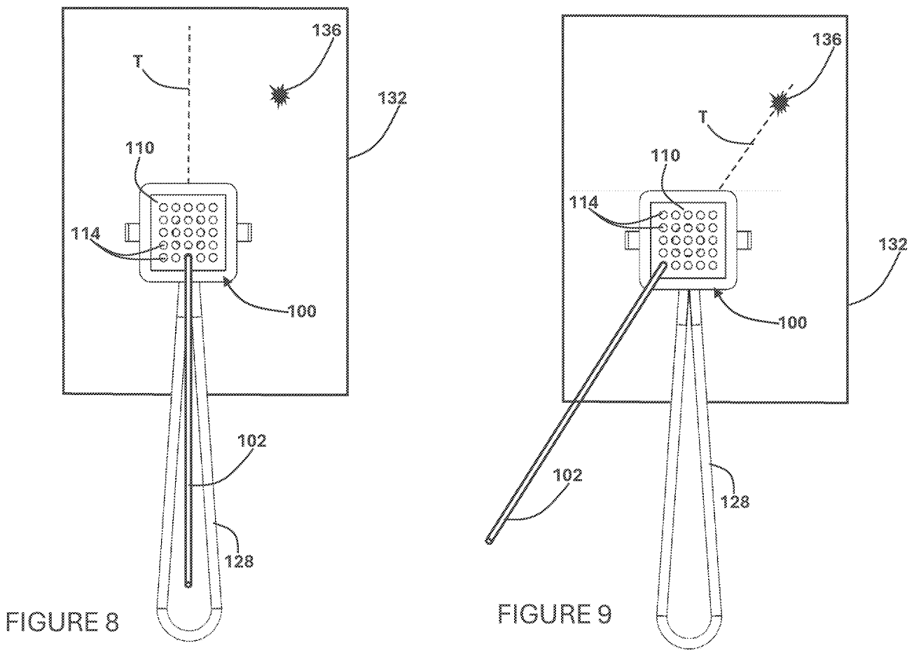

Referring now to FIG. 8, after the desired trajectory T is achieved in the first plane, the X-Ray unit may be moved to a second plane that is perpendicular to the first plane, to assess the trajectory T in the second plane. It can be seen in FIG. 8 that although the desired trajectory T was achieved in the first plane, the position of the guide pin 102 does not provide the desired trajectory T in the second plane, as the trajectory T of the guide pin 102 does not intersect the target location 136 (i.e. the guide pin 102 does not point towards the target location 136).

Referring to FIG. 9, in order to adjust the trajectory T in the second plane, the user may remove the guide pin 102 from the bone 132 and from the surgical tool 100, and re-insert the guide pin 102 through the surgical tool 100. In particular, the user may pass the guide pin 102 through a third of the apertures 114 of the trajectory adjustment plate 110 and into the lumen 116 of the conduit 104 (not visible in FIGS. 8 and 9); through the lumen 116 and into the passage 124 of the bone anchoring tip 106 (not visible in FIGS. 8 and 9); and through the passage 124 and into the pilot hole 134 (not visible in FIGS. 8 and 9) of the bone 132. Alternatively (not shown), the user may leave the guide pin 102 and surgical tool 100 in place, and remove the trajectory adjustment plate 110 from the surgical tool 100 and guide pin 102. The user may then reposition the trajectory adjustment plate 110 such that the guide pin 102 is inserted through the third of the apertures, and reattach the trajectory adjustment plate 110 to the surgical tool 100.

The third of the apertures 114 may be selected by the user; however, in the example shown, the third of the apertures 114 is located in a bottom left corner of the trajectory adjustment plate 110, and is horizontally aligned with the second of the apertures 114, such that the repositioning from the second of the apertures 114 to the third of the apertures 114 adjusts the trajectory T in only the second plane. In other words, the second of the apertures 114 is spaced from the first of the apertures in a first direction (i.e. vertically), and the third of the apertures 114 is spaced from the second of the apertures 114 in a second direction that is transverse to the first direction (i.e. horizontally). As shown in FIG. 9, this repositioning results in the trajectory T of the guide pin 102 pointing generally towards the right as compared to the trajectory T shown in FIG. 8.

The position of the guide pin 102 may then be re-assessed using fluoroscopy, to re-assess the trajectory T of the guide pin 102 into the bone 132 in the second plane. When the guide pin 102 is in the position shown in FIG. 9 and is viewed under fluoroscopy, the user would determine that the guide pin 102 does provide the desired trajectory T in the second plane, as the trajectory T intersects the target location 136. In other words, under fluoroscopy, it would be apparent to the user that the guide pin 102 points towards the target location 136; thus, if a bone screw were inserted over the guide pin 102 to follow the trajectory T of the guide pin as shown in FIG. 9, the bone screw would be in the correct position.

The steps of re-positioning the guide pin 102 and re-assessing the position of the guide pin 102 in the second plane may be repeated as many times as necessary, until the guide pin 102 provides the desired trajectory T in the second plane.

When the guide pin 102 is in the desired position and provides the desired trajectory T, the guide pin is advanced through the surgical tool across the injured structures into the desired termination position. The surgical tool 100 is then removed (by sliding it away from the patient and off of the guide pin 102), and the surgery may proceed by measuring the length of the guide pin within the bone and then implanting the appropriate bone screw or other hardware across the injured tissues over the guide pin 102 to the desired termination position.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A surgical tool for trajectory adjustment in guide pin insertion, comprising:
a conduit extending longitudinally from a proximal end to a distal end, wherein the conduit defines a lumen that extends from a proximal opening at the proximal end to a distal opening at the distal end;
a bone anchoring tip at the distal end of the conduit, wherein the bone anchoring tip has a passage that extends longitudinally therethrough and that is open to the lumen; and
a trajectory adjustment plate at the proximal end of the conduit and at least partially covering the proximal opening, the trajectory adjustment plate comprising a plurality of laterally spaced apart apertures that extend therethrough and that are open to the lumen;
whereby a guide pin is insertable through any one of the apertures into the lumen and from the lumen through the passage, and whereby a trajectory of the guide pin on exit from the passage is adjustable by selecting which one of the apertures through which to insert the guide pin.

2. The surgical tool of claim 1, wherein the apertures are arranged in a grid.

3. The surgical tool of claim 1, wherein the apertures all extend parallel to each other.

4. The surgical tool of claim 1, wherein the bone anchoring tip comprises a plurality of distally extending teeth for engaging with a bone surface.

5. The surgical tool of claim 1, wherein the conduit comprises at least a first radiopaque marker to facilitate positioning of the surgical tool using fluoroscopy.

6. The surgical tool of claim 5, wherein the conduit comprises at least a portion that is generally polygonal in transverse section to define at least a first surface that is generally planar, wherein the first surface provides the first radiopaque marker.

7. The surgical tool of claim 1, wherein the conduit is fabricated from a plastic, the trajectory adjustment plate is fabricated from a metal, and/or the bone anchoring tip is fabricated from a metal.

8. A kit of parts for guide pin insertion, comprising:
a surgical tool comprising (i) a conduit extending longitudinally from a proximal end to a distal end, wherein the conduit defines a lumen that extends from a proximal opening at the proximal end to a distal opening at the distal end, (ii) a bone anchoring tip at the distal end of the conduit, wherein the bone anchoring tip has a passage that extends longitudinally therethrough and that is open to the lumen, and (iii) a trajectory adjustment plate at the proximal end of the conduit and at least partially covering the proximal opening, the trajectory adjustment plate comprising a plurality of spaced apart apertures that extend therethrough and that are open to the lumen; and
a guide pin that is insertable through any one of the apertures into the lumen and from the lumen through the passage, whereby a trajectory of the guide pin on exit from the passage is adjustable by selecting which one of the apertures through which to insert the guide pin.

9. The kit of parts of claim 8, wherein the apertures are arranged in a grid.

10. The kit of parts of claim 8, wherein the apertures all extend parallel to each other.

11. The kit of parts of claim 8, wherein the bone anchoring tip comprises a plurality of distally extending teeth for engaging with a bone surface.

12. The kit of parts of claim 8, wherein the conduit comprises at least a first radiopaque marker to facilitate positioning of the surgical tool using fluoroscopy.

13. The kit of parts of claim 12, wherein the conduit comprises at least a portion that is generally polygonal in transverse section to define at least a first surface that is generally planar, wherein the first surface provides the first radiopaque marker.

14. The kit of parts of claim 8, wherein the conduit is fabricated from a plastic, the trajectory adjustment plate is fabricated from a metal, and/or the bone anchoring tip is fabricated from a metal.

15. A method for adjusting a trajectory of a guide pin, comprising:
a. positioning a bone anchoring tip of a surgical tool against a bone;
b. concurrently with or after step a., inserting a guide pin longitudinally through the surgical tool by positioning the guide pin such that the guide pin extends through a first aperture of a trajectory adjustment plate of the surgical tool and into a lumen of a conduit of the surgical tool, through the lumen and into the bone anchoring tip, and through a passage of the bone anchoring tip and into the bone;
c. using fluoroscopy to assess a trajectory of the guide pin into the bone; and
d. adjusting the trajectory by repositioning the guide pin through a second aperture in the trajectory adjustment plate, whereby the second aperture is spaced laterally from the first aperture.

16. The method of claim 15, wherein step d. comprises (i) removing the guide pin from the bone and the surgical tool, and re-inserting the guide pin through the surgical tool by passing the guide pin through the second aperture of the trajectory adjustment plate and into the lumen, through the lumen and into the passage of the bone anchoring tip, and through the passage and into the bone, or (ii) removing the trajectory adjustment plate from the surgical tool and from the guide pin, repositioning the trajectory adjustment plate such that the guide pin is inserted through the second aperture of the trajectory adjustment plate, and reattaching the trajectory adjustment plate to the surgical tool.

17. The method of claim 15, wherein step a. comprises engaging distal teeth of the bone anchoring tip with a surface of the bone.

18. The method of claim 15, further comprising: e. using fluoroscopy to re-assess the trajectory of the guide pin into the bone; and f. re-adjusting the trajectory by removing the guide pin from the bone and the surgical tool, and re-inserting the guide pin through the surgical tool by passing the guide pin through a third aperture of the trajectory adjustment plate and into the lumen, through the lumen and into the passage of the bone anchoring tip, and through the passage and into the bone, whereby the third aperture is spaced laterally from the first aperture and second aperture.

19. The method of claim 18, wherein step c. comprises assessing the trajectory of the guide pin in a first plane, and step e. comprises re-assessing the trajectory of the guide pin in a second plane that is perpendicular to the first plane.

20. The method of 18, wherein the second aperture is spaced from the first aperture in a first direction, and the third aperture is spaced from the second aperture in a second direction that is transverse to the first direction.
